(19) United States
(12) Patent Application Publication
(10) Pub. No.: US 2023/0151329 A1
(43) Pub. Date: May 18, 2023

(54) FIBROBLAST-BASED IMMUNOTHERAPY OF GRAVES DISEASE

(71) Applicant: FIGENE, LLC, Houston, TX (US)

(72) Inventors: Thomas ICHIM, San Diego, CA (US); Pete O'HEERON, Houston, TX (US)

(21) Appl. No.: 17/754,807

(22) PCT Filed: Oct. 14, 2020
(86) PCT No.: PCT/US2020/055641

§ 371 Date: Apr. 13, 2022

Related U.S. Application Data
(60) Provisional application No. 62/915,152, filed on Oct. 15, 2019.

(51) Int. Cl. C12N 5/077; C12N 5/0784; C07K 14/52; A61P 37/06

(52) U.S. Cl. CPC C12N 5/0656; A61P 37/06; C07K 14/52; C12N 5/0639; C12N 2501/22; C12N 2501/31; C12N 2501/231; C12N 2501/2304

(57) ABSTRACT

Disclosed are means, methods and compositions of matter for treatment of Graves’ Disease using non-modified and/or modified fibroblasts for promotion of immunological tolerance, and/or stimulation of antigen-specific tolerance. In some embodiments, fibroblasts are administered together with antigens associated with Graves’ Disease such as the thyrotropin receptor protein and/or peptides and/or altered peptide ligands derived thereof. In some embodiments, co-administration refers to administration simultaneously or within temporal proximity of each other. In some embodiments, co-administration refers to loading of fibroblasts with antigens and/ or epitopes of antigens associated with Graves’ Disease.

This application claims priority to U.S. Provisional Pat. Application Serial No. 62/915,152, filed Oct. 15, 2019, which is incorporated by reference herein in its entirety.

### TECHNICAL FIELD

Embodiments of the disclosure include at least the fields of cell biology, molecular biology, biochemistry, immunology, physiology, and medicine.

### BACKGROUND

The immune system plays a fundamental role in protecting the body from foreign invaders as well as cleaning damaged or cancer transferred cells within the body. It is fundamentally important for the immune system to differentiate between “self”, meaning what should not be attacked, and “non-self” or “altered-self”, which needs to be destroyed. The cells responsible for the specificity of the immune system are referred to as lymphocytes. Lymphocytes are a class of white blood cells. The antigen-specific immune system comprises a variety of differentiated T cells (thymus-derived lymphocytes) and B cells (bone-marrow-derived lymphocytes). Different categories and sub-categories of lymphocytes are defined by expression of different cell-surface antigens. Specifically, various categories and sub-categories of T cells have been identified by characteristic patterns of cell- surface antigen expression.

It is known that there are many different types of autoimmune diseases that each affect the body in different ways. For example, the autoimmune reaction is directed against the brain in multiple sclerosis and the gut in Crohn’s disease. In other autoimmune diseases such as systemic lupus erythematosus, affected tissues and organs may vary among individuals with the same disease. Ultimately, damage to certain tissues by the immune system may be permanent, as with destruction of insulin-producing cells of the pancreas in Type I diabetes mellitus.

While the incidence of most individual autoimmune diseases is rare, as a group, autoimmune diseases afflict millions of Americans. Most autoimmune diseases strike women more often than men; in particular, they affect women of working age and during their childbearing years.

It is recognized that in a number of autoimmune diseases including, for example, Graves’ disease (GD), Rheumatoid Arthritis (RA), myasthenia gravis, insulin-resistant diabetes (Type 1), antibodies to cell membrane receptors lead to anti-receptor hypersensitivity reactions that alter cellular function as a result of the binding of antibody to membrane receptors, which can have a stimulatory or a blocking effect. For example, in animal models of myasthenia gravis, the production of antibodies by immunization to the acetylcholine receptor has resulted in the typical muscle fatigue and weakness noted in affected humans. This antibody has been shown to be present in serum and on muscle membranes and, further, prevents the binding of endogenously produced acetylcholine to its receptor, thereby preventing muscle activation. Similarly, in some diabetic individuals with extreme insulin resistance, antibodies to insulin receptors have been shown that prevent the binding of insulin to its receptor.

Graves’ disease (GD) is a systemic autoimmune process characterized by several immune system abnormalities, including the production of IgG directed against the thyrotropin receptor, expansion of CD45RO+ T cells and lymphocytic infiltration of the thyroid and connective tissue of the orbit. Thyroid-associated ophthalmopathy (TAO) represents the orbital manifestation of GD. Extra-ocular muscles and fat expand, become inflamed and are remodeled extensively. Cytokines and lipid mediators, synthesized by infiltrating T lymphocytes, monocytes and mast cells, drive tissue remodeling, including the accumulation of hyaluronan, an abundant non-sulfated glycosaminoglycan.

The pathology of the orbital fibroblasts and their exaggerated responses to cytokines such as IL-1β represent the basis for disease susceptibility of these tissues. Why immunocompetent cells are recruited to the orbit in TAO remains uncertain. For GD, the mechanism through which immunocompetent cells are trafficked to affected tissues is critical to understanding and, ultimately, to developing therapies that address both the glandular as well as the non-glandular manifestations of Graves’ Disease.

The antigen recognized by the immune system in GD is the thyroid stimulating hormone, also known as thyrotropin, (TSH) receptor (TSHR).

TSH receptor is one of a family of glycoprotein-coupled hormone receptors, and was cloned in 1990. The TSHR is indispensable for TSH signal transduction, production of thyroid hormone and Tg, and proliferation of thyroid follicular cells. TSHR consists of an extracellular domain (ECD: amino acids 1-418), a seven transmembrane domain (7TMD: 418-683) and an intracellular domain. ECD is also divided into Leucine-rich repeat domain (LRR: 1-276) and a hinge region (277-418).

The induction of tolerance in Graves’ Disease requires immune modulation and reprogramming of the immune response to TSHR from pathological Th17/inflammatory response to Treg based and/or anergy. Means of accomplishing this are not currently available in the art.

### BRIEF SUMMARY

The present disclosure is directed to methods useful for the treatment or prevention of an autoimmune disease, including Graves’ disease. Certain embodiments concern a method or methods of treating an individual having, or suspected of having, Graves’ Disease. Certain embodiments concern the administration of one or more cellular therapies, including wherein the cells comprising the cellular therapy have been enhanced for immune regulatory activity, in combination with one or more peptides and/or proteins, to an individual having, or suspected of having, Graves’ Disease. The therapy may treat one or more symptoms, or may delay the onset and/or reduce the severity of one or more symptoms, related to Graves’ Disease.

The cellular therapy may comprise fibroblasts, which may or may not be modified, activated, dedifferentiated, and/or reprogrammed. The fibroblasts may be from any source including from tissue selected from the group consisting of dermal, adipose, omental, cord blood, Wharton’s Jelly, placental, endometrial, mobilized peripheral blood, bone marrow, peripheral blood, and a combination thereof. In some embodiments, the fibroblasts proliferate at a rate of 14-21 hours per cell multiplication. In some embodiments, the fibroblasts secrete 0.1 pg to 77 pg, or any range derivable therein, of interleukin-1 per culture of 1 million fibroblasts at 75% confluence on a surface. In some embodiments, the fibroblasts secrete 1 pg to 500 pg, or any range derivable therein, of FGF-1 per culture of 1 million fibroblasts at 75% confluence on a surface. In some embodiments, the fibroblasts substantially decrease, such as by more than 20%, the ability of responding T cells to proliferate in a mixed lymphocyte reaction when compared to a control mixed lymphocyte reaction in which fibroblasts are not added.

In some embodiments, the fibroblasts are modified and/or cultured to enhance the immune regulatory activity of the fibroblasts. The fibroblasts may be cultured with or administered hCG and/or oxytocin, such as to enhance the immune regulatory activity of the fibroblasts. In some embodiments, the fibroblasts are cultured with or administered 1 nM hCG per million fibroblasts to 1 µM hCG per million fibroblasts, or any range derivable therein, or 10 nM hCG per million fibroblasts to 100 nM hCG per million fibroblasts, or any range derivable therein. In some embodiments, the fibroblasts are cultured with or administered 1 nM oxytocin per million fibroblasts to 10 µM oxytocin per million fibroblasts, or any range derivable therein, or 100 nM oxytocin per million fibroblasts to 1 µM oxytocin per million fibroblasts, or any range derivable therein. In some embodiments, the fibroblasts are modified, such as by transfection, transduction, or electroporation, for example, to express one or more Graves’ Disease-specific autoantigens. In some embodiments, the fibroblasts may be modified, such as by transfection, transduction, or electroporation, for example, to exogenously express interleukin-10. Any method for exogenously expressing interleukin-10 may be used.

The peptides and/or proteins administered in combination with the cellular therapy may comprise one or more antigens that are present on cells involved in Graves’ Disease. The one or more peptides and/or proteins may comprise Graves’ Disease-specific autoantigens, such as a thyrotropin (TSH) receptor protein or any peptide derived from the TSH receptor protein (including any immunogenic peptide) or altered peptide ligand (peptide with substitutions, including random or conservative, to increase affinity to MHC) derived from the TSH receptor protein. In some embodiments, the proteins and/or peptides are derived from thyroid tissue, cell lysate from thyroid tissue, and/or exosomes from thyroid tissue. In some embodiments, the protein and/or peptide, including those comprising the thyrotropin receptor, stimulates, or is capable of stimulating, T regulatory cells, including Th3 cells (that may produce, or be capable of producing, TGF-beta upon activation). Activation of the Th3 cell may comprise ligation of the T cell receptor. In some embodiments, the thyrotropin receptor protein and/or peptide, including immunogenic and/or altered peptide ligand, derived from the thyrotropin receptor protein reduces, or is capable of reducing, production of IL-17 by Th17 cells.

In specific embodiments, a TSH protein such as is disclosed in GenBank® Accession Number AAA36783.1 is utilized for its sequence. An entire TSH protein may be utilized in methods and compositions of the disclosure, or functional fragments or derivatives may be utilized. In specific embodiments, peptides from the TSH protein are utilized, and the peptides may be of any suitable length. In specific embodiments, the peptide lengths are 8-36 amino acids. The peptide lengths may be 8-36, 8-30, 8-25, 8-20, 8-15-8-10, 10-36, 10-30, 10-25, 10-20, 10-15, 15-36, 15-30, 15-25, 15-20, 20-36, 20-30, 20-25, 25-36, 25-30, or 30-36 amino acids in length, as examples. In specific embodiments, the peptide length is 8, 9, 10, 11, 12, 13, 14, 15, 16, 17, 18, 19, 20, 21, 22, 23, 24, 25, 26, 27, 28, 29, 30, 31, 32, 33, 34, 35, or 36 amino acids in length. The peptides may be derived from any location within the protein, including from the N-terminus or C-terminus, for example. The peptide may or may not be selected randomly. The peptide may come from within TSH amino acids 1-100, 101-200, 201-300, 301 -400, 401-500, 501-600, 601-700, or 701 to the end of the protein, or any range derivable therein any of these ranges. In some cases, the peptide has the aforementioned lengths but is a derivative of the corresponding region in the wild-type TSH protein, such as being 99, 98, 97, 96, 95, 94, 93, 92, 91, 90, 85, 80, 75, or 70% identical to the corresponding region in the wild-type TSH protein.

In some embodiments, one or more Graves’ Disease-specific autoantigens are pulsed into the fibroblasts of the present disclosure, such as by coculturing the fibroblasts with the Graves’ Disease-specific autoantigen(s). In some embodiments, the fibroblasts of the present disclosure and one or more Graves’ Disease-specific autoantigens are administered with immature dendritic cells to an individual. The immature dendritic cells may be generated by any method known in the art including culturing monocytes and/or CD34⁺ cells with IL-4 and GM-CSF. The immature dendritic cells may be cultured with interleukin-10 to maintain their immature state. The immature dendritic cells may be modified, such as by transfection, transduction, or electroporation, for example, to exogenously express interleukin-10. The immature dendritic cells may be cultured with the fibroblasts.

Certain embodiments of the present disclosure concern administering a cellular therapy, one or more Graves’ Disease-specific autoantigens, and optionally immature dendritic cells to an individual in need thereof. The combination may be administered via any suitable route, including a gastrointestinal route, as one example. In some embodiments, the cellular therapy, one or more Graves’ Disease-specific autoantigens, and optionally immature dendritic cells, are administered with one or more immune inhibitor cytokines, including for example TGF-beta, IL-10, and/or IL-35, to an individual. The combination may be administered via any suitable route, including a gastrointestinal route, as one example.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures.

### DETAILED DESCRIPTION

### I. Definitions

As used herein, the term “immune cell” includes cells that are of hematopoietic origin and that play a role in the immune response. Immune cells include lymphocytes, such as B cells and T cells; natural killer cells; and myeloid cells, such as monocytes, macrophages, eosinophils, mast cells, basophils, and granulocytes.

As used herein, the term “T cell” includes CD4+ T cells and CD8+ T cells. The term T cell also includes both T helper 1 type T cells and T helper 2 type T cells.

The term “antigen presenting cell” includes professional antigen presenting cells (e.g., B lymphocytes, monocytes, dendritic cells, and Langerhans cells) as well as other antigen presenting cells (e.g., keratinocytes, endothelial cells, astrocytes, fibroblasts, and oligodendrocytes).

As used herein, the term “immune response” includes T cell-mediated and/or B cell-mediated immune responses that are influenced by modulation of T cell costimulation. Immune responses include B cell responses (e.g., antibody production) T cell responses (e.g., cytokine production, and cellular cytotoxicity) and activation of cytokine responsive cells, e.g., macrophages.

As used herein, the term “costimulatory receptor” includes receptors which transmit a costimulatory signal to an immune cell, e.g., CD28 or ICOS. As used herein, the term “inhibitory receptors” includes receptors which transmit a negative signal to an immune cell

As used herein, the term “costimulate”, with reference to activated immune cells, includes the ability of a costimulatory molecule to provide a second, non-activating, receptor-mediated signal (a “costimulatory signal”) that induces proliferation or effector function. For example, a costimulatory signal can result in cytokine secretion, e.g., in a T cell that has received a T cell-receptor-mediated signal. Immune cells that have received a cell receptor-mediated signal, e.g., via an activating receptor, are referred to herein as “activated immune cells.” An inhibitory signal as transduced by an inhibitory receptor can occur even if a costimulatory receptor (such as CD28 or ICOS) in not present on the immune cell and, thus, is not simply a function of competition between inhibitory receptors and costimulatory receptors for binding of costimulatory molecules (Fallarino et al. (1998) J. Exp. Med. 188:205). Transmission of an inhibitory signal to an immune cell can result in unresponsiveness, anergy or programmed cell death in the immune cell. Preferably, transmission of an inhibitory signal operates through a mechanism that does not involve apoptosis.

As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory molecule) results in failure to produce cytokines and, thus, failure to proliferate. Anergic T cells can, however, mount responses to unrelated antigens and can proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer.

As used herein, the term “tolerogens” or “tolerogen” refers to one or more molecules for which immunologic tolerance is desired, which may be presented, in methods disclosed herein, in combination with the dendritic cells and/or fibroblasts described herein in order to induce stable, long-lasting tolerance, e.g. for greater than about one week, greater than about two weeks, greater than about three weeks, greater than about one month, or more. One or more tolerogens may comprise one or more Graves’ Disease-specific autoantigens.

### II. Fibroblasts and Uses and Preparation Thereof

Certain embodiments of the disclosure concern the use of fibroblasts to induce tolerance to one or more antigens associated with Graves’ Disease. In some embodiments, Fibroblasts are modified, such as gene-modified, to express Graves’ Disease-specific autoantigens, including a thyrotropin receptor (TSHR) protein and/or fragments thereof. The Graves’ Disease-specific autoantigens may be introduced intracellularly into the fibroblasts, such as by transduction, including by means of a protein transduction domain; electroporation; and/or transfection, such as a lipid based transfection. In certain embodiments, the fibroblasts are administered with one or more immune inhibitor cytokines such as TGF-beta and/or IL-10 and/or IL-35. In certain embodiments, the fibroblasts are made into immune inhibitor cells.

A method of treating an individual having, or suspected of having, Graves’ Disease comprising administering fibroblasts and one or more Graves’ disease-specific autoantigens to the individual, wherein immune regulatory activity of the fibroblasts is optionally enhanced. Example routes of administration of fibroblasts and/or one or more Graves’ disease-specific autoantigens and/or dendritic cells include parenteral (e.g., intravenous, intradermal, microvascular bed of bone marrow, subcutaneous), oral (e.g., ingestion or inhalation), transdermal (e.g., topical), transmucosal, and rectal administration. In certain particular aspects, cells are administered from a route selected from a group consisting of: intravenously, intraarterially, intramuscularly, subcutaneously, transdermally, intratracheally, intraperitoneally, intravitreally, and via direct injection into bone compartments or into spinal fluid.

The fibroblasts may be from any source including from tissue selected from the group consisting of dermal, adipose, omental, cord blood, Wharton’s Jelly, placental, endometrial, mobilized peripheral blood, bone marrow, peripheral blood, and a combination thereof. In some embodiments, the fibroblasts proliferate at a rate of 14-21 hours per cell multiplication. In some embodiments, the fibroblasts secrete 0.1 pg to 77 pg, or any range derivable therein, of interleukin-1 per culture of 1 million fibroblasts at 75% confluence on a surface. In some embodiments, the fibroblasts secrete 1 pg to 500 pg, or any range derivable therein, of FGF-1 per culture of 1 million fibroblasts at 75% confluence on a surface. In some embodiments, the fibroblasts substantially decrease, such as by more than 20%, the ability of responding T cells to proliferate in a mixed lymphocyte reaction when compared to a control mixed lymphocyte reaction in which fibroblasts are not added.

In some embodiments, fibroblasts are cultured with hCG and/or oxytocin, which may make the fibroblasts immune inhibitor cells. In certain embodiments, the fibroblasts are pulsed with the Graves’ Disease-specific autoantigens, such as by allowing the Graves’ Disease-specific autoantigens to be uptaken by the fibroblasts by endocytosis. The modified fibroblasts may be subsequently administered in a tolerogenic manner. In some embodiments the transfected fibroblasts are administered together with dendritic cells, including immature and/or tolerogenic dendritic cells.

In certain embodiments, the fibroblasts, including the modified and/or unmodified fibroblasts, are administered to an individual. The fibroblasts may be administered with one or more Graves’ Disease-specific autoantigens. The fibroblasts, in combination with (or not in combination with) one or more Graves’ Disease-specific autoantigens, may be administered with other compositions useful to tolerizing the individual to one or more Graves’ Disease-specific autoantigens. The composition may comprise immature dendritic cells and/or immune inhibitor cytokines, such as TGF-beta, IL-10, and/or IL-35, for example.

The fibroblasts encompassed herein may be generated by any method known in the art, including by outgrowth from a biopsy of the recipient’s own skin (in the case of autologous preparations), or skin of healthy donors (for allogeneic preparations), for example. In some embodiments, fibroblasts are used from young donors. In some embodiments, fibroblasts are transfected with genes to allow for enhanced growth and overcoming of the Hayflick limit. Subsequent to isolation of cells, the expansion in culture using standard cell culture techniques may be performed. Skin tissue (dermis and epidermis layers) may be biopsied from a subject’s post-auricular area. In one embodiment, the starting material is composed of three 3-mm punch skin biopsies collected using standard aseptic practices. The biopsies may be collected by a skilled artisan, placed into a vial containing sterile phosphate buffered saline (PBS). The biopsies may be shipped in a 2-8° C. refrigerated shipper back to the manufacturing facility.

In one embodiment, after arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0 +/- 2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Helidelburg, Germany). After digestion, Initiation Growth Media (IMDM, GA, 10% Fetal Bovine Serum (FBS)) is added to neutralize the enzyme, cells may be pelleted by centrifugation and resuspended in 5.0 mL Initiation Growth Media. Alternatively, centrifugation is not performed, with full inactivation of the enzyme occurring by the addition of Initiation Growth Media only. Initiation Growth Media may be added prior to seeding of the cell suspension into a T-175 cell culture flask for initiation of cell growth and expansion. A T-75, T-150, T-185 or T-225 flask may be used in place of the T-75 flask. Cells may be incubated at 37.0 +/- 2.0° C. with 5.0 +/-1.0% CO₂ and fed with fresh Complete Growth Media every three to five days. All feeds in the process may be performed by removing half of the Complete Growth Media and replacing the same volume with fresh media. Alternatively, full feeds can be performed. Cells should not remain in the T-175 flask for more than approximately 30 days prior to passaging. Confluence is monitored throughout the process to ensure adequate seeding densities during culture splitting. When cell confluence is greater than or equal to 40% in the T-175 flask, they may be passaged by removing the spent media, washing the cells, and treating with Trypsin-EDTA to release adherent cells in the flask into the solution. Cells may then be trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask.

Morphology may be evaluated at each passage and prior to harvest to monitor the culture purity throughout the culture purity throughout the process. Morphology may be evaluated by any technique, such as comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes may appear round and irregularly shaped and, at higher confluence, they appear organized in a cobblestone formation. At lower confluence, keratinocytes are observable in small colonies. Cells may be incubated at 37.0 +/-2.0° C. with 5.0 +/- 1.0% CO₂ and passaged every three to five days in the T-500 flask and every five to seven days in the ten layer cell stack (10 CS). Cells typically should not remain in the T-500 flask for more than 10 days prior to passaging. Quality Control (QC) release testing for safety of the Bulk Drug Substance (here, cells) includes sterility and endotoxin testing. When cell confluence in the T-500 flask is >95%, cells are passaged to a 10 CS culture vessel. Alternately, two Five Layer Cell Stacks (5 CS) or a 10 Layer Cell Factory (10 CF) can be used in place of the 10 CS. 10 CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37.0 +/-2.0° C. with 5.0 +/- 1.0% CO₂ and fed with fresh Complete Growth Media every five to seven days. Cells typically should not remain in the 10 CS for more than 20 days prior to passaging. In one embodiment, the passaged dermal fibroblasts are rendered substantially free of immunogenic proteins present in the culture medium by incubating the expanded fibroblasts for a period of time in protein free medium, Typically, when cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

In some embodiments, when large numbers of cells are required after receiving cell count results from the primary 10 CS harvest, an additional passage into multiple cell stacks (up to four 10 CS) is performed. For additional passaging, cells from the primary harvest are added to a 2 L media bottle containing fresh Complete Growth Media. Resuspended cells are added to multiple cell stacks and incubated at 37.0 +/- 2.0° C. with 5.0 +/- 1.0% CO₂. The cell stacks are fed and harvested as described above, except cell confluence must be 80% or higher prior to cell harvest. The harvest procedure is the same as described for the primary harvest above. A mycoplasma sample from cells and spent media is collected, and cell count and viability performed as described for the primary harvest above. The method decreases or eliminates immunogenic proteins be avoiding their introduction from animal-sourced reagents. To reduce process residuals, cells are cryopreserved in protein-free freeze media, then thawed and washed prior to prepping the final injection to further reduce remaining residuals. If additional Drug Substance (here, the cells) is needed after the harvest and cryopreservation of cells from additional passaging is complete, aliquots of frozen Drug Substance--Cryovial are thawed and used to seed 5 CS or 10 CS culture vessels. Alternatively, a four layer cell factory (4 CF), two 4 CF, or two 5 CS can be used in place of a 5 CS or 10 CS. A frozen cryovial(s) of cells is thawed, washed, added to a 2 L media bottle containing fresh Complete Growth Media and cultured, harvested and cryopreserved as described above. The cell suspension is added Cell confluence must be 80% or more prior to cell harvest.

At the completion of culture expansion, the cells are harvested and washed, then formulated to contain approximately 10,000 cells to 2.7 × 10⁹ cells/mL, with a target of 2.2 × 10⁷ cells/mL. Alternatively, the target can be adjusted within the formulation range to accommodate different indication doses. The drug substance may comprise a population of viable, autologous human fibroblast cells suspended in a cryopreservation medium consisting of Iscove’s Modified Dulbecco’s Medium (IMDM) and Profreeze-CDM™ (Lonza, Walkerville, Md.) plus 7.5% dimethyl sulfoxide (DMSO). Alternatively, a lower DMSO concentration may be used in place of 7.5% or CryoStor™ CS5 or CryoStor™ CS10 (BioLife Solutions, Bothell, Wash.) may be used in place of IMDM/Profreeze/DMSO. In addition to cell count and viability, purity/identity of the Drug Substance is performed and must confirm the suspension contains 98% or more fibroblasts. The usual cell contaminants include keratinocytes. The purity/identify assay employs fluorescent-tagged antibodies against CD90 and CD 104 (cell surface markers for fibroblast and keratinocyte cells, respectively) to quantify the percent purity of a fibroblast cell population. CD90 (Thy-1) is a 35 kDa cell-surface glycoprotein. Antibodies against CD90 protein have been shown to exhibit high specificity to human fibroblast cells. CD104, integrin β4 chain, is a 205 kDa transmembrane glycoprotein which associates with integrin α6 chain (CD49f) to form the α6/β4 complex. This complex has been shown to act as a molecular marker for keratinocyte cells (Adams and Watt 1991).

Antibodies to CD 104 protein bind to approximately 100% of human keratinocyte cells. Cell count and viability is determined by incubating the samples with Viacount Dye Reagent and analyzing samples using the Guava PCA system. The reagent is composed of two dyes, a membrane-permeable dye which stains all nucleated cells, and a membrane-impermeable dye which stains only damaged or dying cells. The use of this dye combination enables the Guava PCA system to estimate the total number of cells present in the sample, and to determine which cells are viable, apoptotic, or dead. The method was custom developed specifically for use in determining purity/identity of autologous cultured fibroblasts.

Alternatively, cells can be passaged from either the T-175 flask (or alternatives) or the T-500 flask (or alternatives) into a spinner flask containing microcarriers as the cell growth surface. Microcarriers are small bead-like structures that are used as a growth surface for anchorage-dependent cells in suspension culture. They are designed to produce large cell yields in small volumes. In this apparatus, a volume of Complete Growth Media ranging from 50 mL-300 mL is added to a 500 mL, IL or 2 L sterile disposable spinner flask. Sterile microcarriers are added to the spinner flask. The culture is allowed to remain static or is placed on a stir plate at a low RPM (15-30 RRM) for a short period of time (1-24 hours) in a 37 +/- 2.0° C. with 5.0 +/-1.0% CO₂ incubator to allow for adherence of cells to the carriers. After the attachment period, the speed of the spin plate is increased (30-120 RPM). Cells are fed with fresh Complete Growth Media every one to five days, or when media appears spent by color change. Cells are collected at regular intervals by sampling the microcarriers, isolating the cells and performing cell count and viability analysis. The concentration of cells per carrier is used to determine when to scale-up the culture. When enough cells are produced, cells are washed with PBS and harvested from the microcarriers using trypsin-EDTA and seeded back into the spinner flask in a larger amount of microcarriers and higher volume of Complete Growth Media (300 mL-2 L). Alternatively, additional microcarriers and Complete Growth Media can be added directly to the spinner flask containing the existing microcarrier culture, allowing for direct bead-to-bead transfer of cells without the use of trypsinization and reseeding. Alternatively, if enough cells are produced from the initial T-175 or T-500 flask, the cells can be directly seeded into the scale-up amount of microcarriers. After the attachment period, the speed of the spin plate is increased (30-120 RPM). Cells are fed with fresh Complete Growth Media every one to five days, or when media appears spent by color change. When the concentration reaches the desired cell count for the intended indication, the cells are washed with PBS and harvested using trypsin-EDTA. Microcarriers used within the disposable spinner flask may be made from poly blend such as BioNOC II® (Cesco Bioengineering, distributed by Bellco Biotechnology, Vineland, N.J.) and FibraCel® (New Brunswick Scientific, Edison, N.J.), gelatin, such as Cultispher-G (Percell Biolytica, Astrop, Sweden), cellulose, such as Cytopore™ (GE Healthcare, Piscataway, N.J.) or coated/uncoated polystyrene, such as 2D MicroHex™ (Nunc, Weisbaden, Germany), Cytodex® (GE Healthcare, Piscataway, N.J.) or Hy-Q Sphere™ (Thermo Scientific Hyclone, Logan, Utah).

In another embodiment, cells can be processed on poly blend 2D microcarriers such as BioNOC II® and FibraCel® using an automatic bellow system, such as FibraStage.TM. (New Brunswick Scientific, Edison, N.J.) or BelloCell® (Cesco Bioengineering, distributed by Bellco Biotechnology, Vineland, N.J.) in place of the spinner flask apparatus. Cells from the T-175 (or alternatives) or T-500 flask (or alternatives) are passaged into a bellow bottle containing microcarriers with the appropriate amount of Complete Growth Media, and placed into the system. The system pumps media over the microcarriers to feed cells, and draws away media to allow for oxygenation in a repeating fixed cycle. Cells are monitored, fed, washed and harvested in the same sequence as described above. Alternatively, cells can be processed using automated systems. After digestion of the biopsy tissue or after the first passage is complete (T-175 flask or alternative), cells may be seeded into an automated device. One method is an Automated Cellular Expansion (ACE) system, which is a series of commercially available or custom fabricated components linked together to form a cell growth platform in which cells can be expanded without human intervention. Cells are expanded in a cell tower, consisting of a stack of disks capable of supporting anchorage-dependent cell attachment. The system automatically circulates media and performs trypsinization for harvest upon completion of the cell expansion stage.

Alternatively, the ACE system can be a scaled down, including as a single lot unit version comprised of a disposable component that consists of cell growth surface, delivery tubing, media and reagents, and a permanent base that houses mechanics and computer processing capabilities for heating/cooling, media transfer and execution of the automated programming cycle. Upon receipt, each sterile irradiated ACE disposable unit will be unwrapped from its packaging and loaded with media and reagents by hanging pre-filled bags and connecting the bags to the existing tubing via aseptic connectors. The process continues as follows: a) Inside a biological safety cabinet (BSC), a suspension of cells from a biopsy that has been enzymatically digested is introduced into the “pre-growth chamber” (small unit on top of the cell tower), which is already filled with Initiation Growth Media containing antibiotics. From the BSC, the disposable would be transferred to the permanent ACE unit already in place; b) After approximately three days, the cells within the pre-growth chamber are trypsinized and introduced into the cell tower itself, which is pre-filled with Complete Growth Media. Here, the “bubbling action” caused by CO₂ injection force the media to circulate at such a rate that the cells spiral downward and settle on the surface of the discs in an evenly distributed manner; c) For approximately seven days, the cells are allowed to multiply. At this time, confluence will be checked (method unknown at time of writing) to verify that culture is growing. Also at this time, the Complete Growth Media will be replaced with fresh Complete Growth Media. CGM will be replaced every seven days for three to four weeks. At the end of the culture period, the confluence is checked once more to verify that there is sufficient growth to possibly yield the desired quantity of cells for the intended treatment; d) If the culture is sufficiently confluent, it is harvested. The spent media (supernatant) is drained from the vessel. PBS will then is pumped into the vessel (to wash the media, FBS from the cells) and drained almost immediately. Trypsin-EDTA is pumped into the vessel to detach the cells from the growth surface. The trypsin/cell mixture is drained from the vessel and enter the spin separator. Cryopreservative is pumped into the vessel to rinse any residual cells from the surface of the discs, and be sent to the spin separator as well. The spin separator collects the cells and then evenly resuspend the cells in the shipping/injection medium. From the spin separator, the cells will be sent through an inline automated cell counting device or a sample collected for cell count and viability testing via laboratory analyses. Once a specific number of cells has been counted and the proper cell concentration has been reached, the harvested cells are delivered to a collection vial that can be removed to aliquot the samples for cryogenic freezing.

In another embodiment, automated robotic systems may be used to perform cell feeding, passaging, and harvesting for the entire length or a portion of the process. Cells can be introduced into the robotic device directly after digest and seed into the T-175 flask (or alternative). The device may have the capacity to incubate cells, perform cell count and viability analysis and perform feeds and transfers to larger culture vessels. The system may also have a computerized cataloging function to track individual lots. Existing technologies or customized systems may be used for the robotic option.

In some embodiments, fibroblasts are preactivated by contact with a growth factor containing mixture; the mixture or composition may comprise one or more growth factors selected from the group consisting of transforming growth factors (TGF), fibroblast growth factors (FGF), platelet-derived growth factors (PDGF), epidermal growth factors (EGF), vascular endothelial growth factors (VEGF), insulin-like growth factors (IGF), platelet-derived endothelial growth factors (PDEGF), platelet-derived angiogenesis factors (PDAF), platelet factors 4 (PF-4), hepatocyte growth factors (HGF) and mixtures thereof. In some cases, the growth factors are transforming growth factors (TGF), platelet-derived growth factors (PDGF) fibroblast growth factors (FGF) or mixtures thereof. In certain cases, the growth factors are selected from the group consisting of transforming growth factors β (TGF-β), platelet-derived growth factors BB (PDGF-BB), basic fibroblast growth factors (bFGF) and a combination thereof. In some embodiments, said growth factor containing compositions are injected simultaneously with, or subsequent to, injection of fibroblasts. Said fibroblasts may be autologous, allogeneic, or xenogeneic.

In some embodiments, a platelet plasma composition is administered together with the fibroblasts or subsequent to administration of the fibroblasts, the composition, comprises, consists essentially of, or consists of platelets and/or plasma and may be derived from bone marrow and/or peripheral blood. Certain embodiments of the disclosure concern the use of platelet plasma compositions from either or both of these sources, and either platelet plasma composition may be used to regenerate either a nucleus or annulus in need thereof. Further, the platelet plasma composition may be used with or without concentrated bone marrow (BMAC). Platelets are non-nucleated blood cells that as noted above are found in bone marrow and peripheral blood. They have several important functions such as controlling bleeding and tissue healing. As persons of ordinary skill in the art are aware, the ability to promote tissue healing is due to the many growth factors that they produce including platelet-derived growth factor (PDGF), transforming growth factor beta (TGF-beta), fibroblast growth factor (FGF), insulin-like growth factor-1 (IGF-1), connective tissue growth factor (CTGF) and vascular endothelial growth factor (VEGF). Many of these platelet proteins and molecules are cytokines and are important for cell signaling and immunomodulation.

### III. Graves’ Disease-Specific Autoantigens

As used herein, the term “Graves’ Disease-specific autoantigen” may refer to any macromolecule, such as a protein and/or peptide, that is recognized by the immune system, including immune cells and antibodies, in an individual having, or suspected of having, Graves’ Disease. The Graves’ Disease-specific autoantigen may be expressed on any cell or tissue, including the thyroid. The Graves’ Disease-specific autoantigen may comprise antigens that are recognized by the immune system in diseases or syndromes that are not classified as Graves’ Disease. The Graves’ Disease-specific autoantigen may be an autoantigen that is involved in other diseases, including other autoimmune diseases. The Graves’ Disease-specific autoantigen may comprise the thyrotropin (also known as thyroid stimulating receptor or TSH) receptor protein, or any peptide (or altered peptide ligand) derived from the thyrotropin receptor protein.

Graves’ Disease-specific autoantigens may be any tolerogenic molecules that are specific for Graves’ Disease, used in the methods of this disclosure. The tolerogen, which may be a Graves’ Disease-specific autoantigen, contributes to the specificity of the tolerogenic response that is induced. It may or may not be the same as the target antigen, which is the antigen present or to be placed in the subject being treated which is a target for the unwanted immunological response, and for which tolerance is desired. The Graves’ Disease-specific autoantigen encompassed in methods herein for administering to an individual may or may not be the same autoantigen (i.e. same peptide sequence, nucleic acid sequence, carbohydrate sequence, lipid sequence, or combination thereof) that is targeted by the immune system of the individual that causes or progresses Graves’ Disease. A tolerogen of this disclosure, which may be a Graves’ Disease-specific autoantigen, may be a polypeptide, polynucleotide, carbohydrate, glycolipid, or other molecule isolated from a biological source, or it may be a chemically synthesized small molecule, polymer, or derivative of a biological material, providing it has the ability to induce tolerance according to this description when combined with the mucosal-binding component (for example, for oral tolerance when the antigen is delivered into the oral mucosa or gut mucosa).

In certain embodiments, the tolerogen, which may be a Graves’ Disease-specific autoantigen, is not in the same form as expressed in the individual being treated, but is a fragment or derivative thereof. Tolerogens of these embodiments include peptides based on a molecule of the appropriate specificity but adapted by fragmentation, residue substitution, labeling, conjugation, and/or fusion with peptides having other functional properties. The adaptation may be performed for any desirable purposes, including but not limited to the elimination of any undesirable property, such as toxicity or immunogenicity; or to enhance any desirable property, such as mucosal binding, mucosal penetration, or stimulation of the tolerogenic arm of the immune response. Tolerogenic regions of an inducing antigen may be different from immunodominant epitopes for the stimulation of an antibody response. Tolerogenic regions are generally regions that can be presented in particular cellular interactions involving T cells. Tolerogenic regions may be present and capable of inducing tolerance upon presentation of the intact antigen. Some antigens contain cryptic tolerogenic regions, in that the processing and presentation of the native antigen does not normally trigger tolerance.

In certain embodiments, two, three, or a higher plurality of tolerogens are used. It may be desirable to implement these embodiments when there is a plurality of target antigens. It may also be desirable to provide a cocktail of antigens to cover several possible alternative targets. Tolerogens can be prepared by a number of techniques known in the art, depending on the nature of the molecule. Polynucleotide, polypeptide, and carbohydrate antigens can be isolated from cells of the species to be treated in which they are enriched. Short peptides are conveniently prepared by amino acid synthesis. Longer proteins of known sequence can be prepared by synthesizing an encoding sequence or PCR-amplifying an encoding sequence from a natural source or vector, and then expressing the encoding sequence in a suitable bacterial or eukaryotic host cell.

In certain embodiments, the tolerogen comprises a complex mixture of antigens obtained from a cell or tissue related to Graves’ Disease, one or more of which plays the role of tolerogen, or Graves’ Disease-specific autoantigen encompassed herein. The tolerogens may be in the form of whole cells, either intact or treated with a fixative such as formaldehyde, glutaraldehyde, or alcohol; in the form of a cell lysate, created by detergent solubilization or mechanical rupture of cells or tissue, followed by clarification. The tolerogens may also be obtained by subcellular fractionation, particularly an enrichment of plasma membrane by techniques such as differential centrifugation, optionally followed by detergent solubilization and dialysis. Other separation techniques are also suitable, such as affinity or ion exchange chromatography of solubilized membrane proteins.

In some embodiments, the tolerogen or Graves’ Disease-specific autoantigen comprises the thyrotropin (also known as TSH) receptor protein, or any peptide, immunogenic peptide, functional fragment, or altered peptide ligand derived from the thyrotropin receptor protein.

The disclosure may be used for treatment of a variety of autoimmune conditions by replacing the thyroid antigens or Graves’ Disease-specific autoantigens with antigens selective for the specific autoimmune condition. A large number of conditions have, or likely have, an autoimmune cause or component, including, for example, Graves’ disease, rheumatoid arthritis (RA), Insulin dependent Diabetes (Type I), Alopecia Areata, Ankylosing Spondylitis, Antiphospholipid Syndrome, Autoimmune Addison’s Disease, Autoimmune Hemolytic Anemia, Autoimmune Hepatitis, Behcet’s Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome Cicatricial Pemphigoid, CREST Syndrome, Cold Agglutinin Disease, Crohn’s Disease, Discoid Lupus, Essential Mixed Cryoglobulinemia, Fibromyalgia-Fibromyositis, Guillain-Barre, Hashimoto’s Thyroiditis, Idiopathic Pulmonary Fibrosis, Idiopathic Thrombocytopenia Purpura (ITP), IgA Nephropathy, Juvenile Arthritis, Lichen Planus, Lupus, Meniere’s Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud’s Phenomenon, Reiter’s Syndrome, Rheumatic Fever, Sarcoidosis, Scleroderma, Sjogren’s Syndrome, Stiff-Man Syndrome, Takayasu Arteritis, Temporal Arteritis/Giant Cell Arteritis, Ulcerative Colitis, Uveitis, Vasculitis, Vitiligo, Wegener’s Granulomatosis.

In some embodiments, one or more Graves’ Disease-specific autoantigens are expressed and/or presented on fibroblasts. In some embodiments, one or more Graves’ Disease-specific autoantigens comprise or are present on the cellular membrane of the fibroblasts. Any method known in the art for expressing, presenting, or otherwise translocating one or more Graves’ Disease-specific autoantigens on the cellular membrane of fibroblasts may be used. The fibroblasts may be modified, such as by transfection, transduction, or electroporation of nucleic acids that encode for one or more Graves’ Disease-specific autoantigens. The Graves’ Disease-specific autoantigens may be pulsed into fibroblasts. The fibroblasts expressing, presenting, or comprising Graves’ Disease-specific autoantigens may be used to induce tolerance, including oral tolerance, in an individual. Oral tolerance may be amplified by administration of agents such as low dose IL-2, which has been shown to potentiate tolerogenic processes.

### IV. Dendritic Cells

In some embodiments, fibroblasts are grown together with immature dendritic cells and used to present antigen in a tolerogenic manner. In a specific embodiment, fibroblasts are transfected with autoantigens implicated in Graves’ Disease and utilized as an antigenic source for immature dendritic cells, wherein said immature dendritic cells present said autoantigens in a tolerogenic manner in order to induce and maintain the state of self-tolerance. Means of generating immature dendritic cells are utilization of these cells for induction of tolerogenesis is well known in the art. In one study, Steinbrink et al. assessed the immune modulatory activity of immature DC, harvested on days 9 to 11 and exposed them IL-10 for the last 2 days of culture, show a strongly reduced capacity to stimulate a CD4+ T cell response in an allogeneic MLR in a dose-dependent manner. In contrast, fully mature DC are completely resistant to the effects of IL-10. These results were obtained in both an alloantigen-induced MLR and an anti-CD3 mAb-induced response of primed and naive (CD45RA+) CD4+ T cells. FACS analysis revealed inhibition of the up-regulation of the costimulatory molecules CD58 and CD86 and the specific DC marker CD83 in DC pretreated with IL-10. These data suggest that IL-10 inhibited the development of fully mature DC. Furthermore, DC precultured with IL-10, but not controls, induced a state of alloantigen-specific anergy in CD4+ T cells and of peptide-specific anergy in the influenza hemagglutinin-specific T cell clone HA1.7. Analysis of the supernatants of these anergic T cells revealed a reduced production of IL-2 and IFN-gamma compared with that in control cells. The authors concluded that IL-10 converts immature DC into tolerogenic APC, which might be a useful tool in the therapy of patients with autoimmune or allergic diseases. Other studies have supported the utility of IL-10 in generation of tolerogenic DC, as well as maintaining tolerance/anergy in T cells. Indeed IL-10 has been implicated in numerous conditions of tolerogenesis including transplant tolerance, cancer, parasitic infection, ocular, and testicular, immune privilege, as well as survival of the fetal allograft.

In some embodiments, fibroblasts and/or dendritic cells are modified, such as by transfection, transduction, or electroporation, to exogenously express IL-10. In some embodiments, IL-10 is transfected into fibroblasts and/or DCs, such as to allow for a consistent and constant production of this cytokine. Means of transfecting cells, including dendritic cells, with IL-10 are known in the art.

In some embodiments, fibroblasts are cultured with dendritic cells, wherein said fibroblasts are transfected with one or more autoantigens, including Graves’ Disease-specific autoantigens. The coculture allows the natural uptake of antigens (autoantigens) from fibroblasts into dendritic cells. Said dendritic cells are kept in an immature state in order to promote antigen-specific tolerogenic programs. DCs are highly specialized antigen presenting cells (APC) that classically initiate Ag-specific immune responses upon infection. This process involves the terminal maturation of DC, typically induced by agents associated with microbial infection. It is now clear that DC can be not only immunogenic but also tolerogenic. In steady state DC remains immature DC and can induce tolerance via deletion of Ag-specific effector T cells and/or differentiation of Tr cells. Repetitive stimulation of naive cord blood CD4+ T cells with allogeneic immature DC results in the differentiation of IL-10-producing T regulatory (Tr) cells, which suppress T-cell responses via a cell-contact dependent mechanism. It was reported that peripheral blood naive CD4+ T cells stimulated with allogeneic immature DC become increasingly hypo-responsive to re-activation with mature DC and after three rounds of stimulation with immature DC, they are profoundly anergic and acquire regulatory function. These T cells are phenotypically and functionally similar to Tr1 cells since they secrete high levels of IL-10 and TGF-β, suppress T-cell responses via an IL-10- and TGF-β-dependent mechanism, and their induction can be blocked by anti-IL10 mAb. Not only immature DC but also specialized subsets of tolerogenic DC can drive the differentiation of Tr cells. Maturation and function of DC can be regulated at different levels. Both pharmacological and biological agents have been shown capable of inducing tolerogenic DC. Several biological agents including IL-10 ,TGF-β, IFN-α, and TNF-α can induce Tr cells. The presence of IL-10 during maturation of DC generate tolerogenic DC, which express low levels of costimulatory molecules and MHC class II, display low stimulatory capacity, and induce antigen-specific T cells anergy in both CD4+ and CD8+ T cells.

The clinical utilization of DC for stimulation of antigen-specific immunity is well known. Examples will be provided of various protocols, which are incorporated by reference, for utilization of DC to stimulate immunity. In some embodiments, fibroblasts transfected with autoantigens are utilized to pulse dendritic cells, and administer them in a tolerogenic environment. Below are examples of clinical use of DC. They have been used in the following cancers: melanoma, soft tissue sarcoma, thyroid, glioma, multiple myeloma , lymphoma, leukemia, as well as liver, lung, ovarian, and pancreatic cancer. In other embodiments of the disclosure concern the use of extracorporeal removal of immunological blocking factors for augmentation of existing dendritic cells to infiltrate tumors. Means of assessing dendritic cell infiltration are known in the art and described in the following examples: for gastric cancer, head and neck cancer, cervical cancer, breast cancer, lung cancer, colorectal cancer, liver cancer, gall bladder cancer, and pancreatic cancer.

According to embodiments of the disclosure, dendritic cells and/or fibroblasts are pulsed with tolerogens, otherwise known as tolerogenic antigens, such as Graves’ Disease-specific autoantigens.

### EXAMPLES

The following examples are included to demonstrate particular embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Treatment or Prevention of Graves’ Disease

An individual having Graves’ Disease or at high risk of having it (e.g., family history, having one or more other autoimmune disorders, being a smoker, etc.) or suspected of having it in specific embodiments is an individual in need thereof. The individual may have been diagnosed by blood test for TSH, by radioactive iodine uptake, ultrasound, imaging tests, or a combination thereof. The treatment may comprise administering an effective amount of fibroblasts and one or more Graves’ disease-specific autoantigens to the individual. Additional treatments may be used, including radioactive iodine therapy, anti-thyroid medications (propylthiouracil and/or methimazole, for example), beta blockers, surgery, or a combination thereof.

In some cases, the fibroblasts have been modified prior to their use, such as being exposed to an effective amount of one or more agents, including agents that promote immunological tolerance and/or stimulate antigen-specific tolerance. In some embodiments, fibroblasts are administered together with one or more antigens associated with Graves’ Disease, such as the thyrotropin receptor protein and/or peptides and/or altered peptide ligands derived thereof. In some embodiments, any cells and the one or more antigens associated with Graves’ Disease are administered substantially simultaneously or within temporal proximity of each other (e.g., within 1-60 seconds, 1-60 minutes, or within 1-24 hours). In some embodiments, the fibroblasts (and/or dendritic cells) are loaded with the antigens and/or epitopes of antigen, such as in the form of peptides, whereas in other cases the fibroblasts (and/or dendritic cells) have been transfected or transduced with exogenous thyrotropin receptor protein or peptides any kind thereof.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of treating an individual having, or suspected of having, Graves’ Disease comprising administering an effective amount of fibroblasts and one or more Graves’ disease-specific autoantigens to the individual.

2. The method of claim 1, wherein the fibroblasts are obtained from tissue selected from the group consisting of dermal, adipose, omental, cord blood, Wharton’s Jelly, placenta, endometrium, mobilized peripheral blood, bone marrow, peripheral blood, and a combination thereof.

3. The method of claims 1 or 2, wherein the fibroblasts proliferate at a rate of 14-21 hours per cell multiplication.

4. The method of any one of claims 1-3, wherein the fibroblasts secrete 0.1 pg-77 pg of interleukin(IL)-1 per culture of 1 million fibroblasts at 75% confluence on a surface.

5. The method of any one of claims 1-4, wherein the fibroblasts secrete 1 pg-500 pg of fibroblast growth factor (FGF-1) per culture of 1 million fibroblasts at 75% confluence on a surface.

6. The method of any one of claims 1-5, wherein the fibroblasts substantially decrease the ability of responding T cells to proliferate in a mixed lymphocyte reaction.

7. The method of claim 6, wherein the decrease in the ability of responding T cells to proliferate comprises a decrease of more than 20% as compared to a control mixed lymphocyte reaction in which fibroblasts are not added.

8. The method of any one of claims 1-7, wherein the fibroblasts have been treated with an effective amount of human chorionic gonadotropin (hCG).

9. The method of claim 8, wherein hCG is administered to the fibroblasts at a concentration of 1 nM to 1 µM.

10. The method of claim 8, wherein hCG is administered to the fibroblasts at a concentration of 10 nM to 100 nM.

11. The method of any one of claims 1-10, wherein the fibroblasts have been treated with an effective amount of oxytocin.

12. The method of claim 11, wherein the oxytocin is administered to the fibroblasts at a concentration of 1 nM to 10 µM.

13. The method of claim 12, wherein the oxytocin is administered to the fibroblasts at a concentration of 100 nM to 1 µM.

14. The method of any one of claims 1-13, wherein the Graves’ Disease-specific autoantigen comprises a thyrotropin receptor protein.

15. The method of claim 14, wherein the Graves’ Disease-specific autoantigen comprises an immunogenic peptide derived from the thyrotropin receptor protein.

16. The method of claim 15, wherein the immunogenic peptide derived from said thyrotropin receptor protein comprises an altered peptide ligand.

17. The method of claim 15, wherein the immunogenic peptide derived from said thyrotropin receptor protein is capable of stimulating expansion of T regulatory cells.

18. The method of any one of claims 15-17, wherein the immunogenic peptide derived from said thyrotropin receptor protein is capable of stimulating expansion of Th3 cells.

19. The method of claim 18, wherein the Th3 cells are capable of producing TGF-beta upon activation.

20. The method of claim 19, wherein the activation of said Th3 cells refers to ligation of T cell receptor.

21. The method of any one of claims 15-20, wherein the immunogenic peptide derived from said thyrotropin receptor protein is capable of reducing production of IL-17 by Th17 cells.

22. The method of any one of claims 1-21, wherein the one or more Graves’ Disease-specific autoantigens are pulsed into the fibroblasts.

23. The method of claim 22, wherein the autoantigen(s) is(are) pulsed into the fibroblasts by co-culturing the autoantigen(s) with the fibroblasts.

24. The method of any one of claims 1-23, wherein the one or more Graves’ Disease-specific autoantigens are derived from cell lysate of thyroid tissue.

25. The method of any one of claims 1-24, wherein the one or more Graves’ Disease-specific autoantigens are derived from exosomes of thyroid tissue.

26. The method of any one of claims 1-25, wherein the fibroblasts are modified to exogenously express IL-10.

27. The method of any one of claims 1-26, wherein the fibroblasts and the one or more Graves’ Disease-specific autoantigens are administered together with an effective amount of immature dendritic cells.

28. The method of claim 27, wherein the immature dendritic cells are generated by culturing monocytes with IL-4 and GM-CSF.

29. The method of claim 27, wherein the immature dendritic cells are generated by culturing CD34 cells with IL-4 and GM-CSF.

30. The method of any one of claims 27-29, wherein the immature dendritic cells are maintained in an immature state by culture with IL-10.

31. The method of any one of claims 27-30, wherein the immature dendritic cells are modified to express exogenous IL-10.

32. The method of any one of claims 27-31, wherein the immature dendritic cells are cultured with the fibroblasts prior to administering the immature dendritic cells, fibroblasts, and one or more Graves’ Disease specific autoantigens to the individual.

33. The method of any one of claims 1-32, wherein the fibroblasts are modified to exogenously express one or more autoantigens.

34. The method of any one of claims 1-33, wherein the fibroblasts and Graves’ Disease-specific autoantigens are administered to the individual together, at substantially the same time, separately, or at different times.

35. The method of claim 34, wherein the administration is a gastrointestinal route.

36. The method of any one of claims 1-35, wherein the fibroblasts and one or more Graves’ Disease-specific autoantigens are administered with one or more immune inhibitor cytokines.

37. The method of claim 36, wherein the immune inhibitor cytokines comprise TGF-beta, IL-10, and/or IL-35.

38. The method of any one of claims 1-37, wherein the individual is provided an effective amount of an additional therapy for Graves’ Disease.
